(19) United States
(12) Patent Application Publication
(10) Pub. No.: US 2022/0254447 A1
(43) Pub. Date: Aug. 11, 2022

(54) INTERMEDIATE RECURRENT PARENTS, AN ACCELERATED AND EFFICIENT MULTI-LAYER TRAIT DELIVERY SYSTEM

(71) Applicant: Monsanto Technology LLC, St, Louis, MO (US)

(72) Inventors: Carrin Carlson, Wildwood, MO (US); Qianni Dong, Maryland Heights, MO (US); Humberto I. Gutierrez, Wildwood, MO (US); Scott Jackson, Bishop, GA (US); Bing Liu, Chesterfield, MO (US); Jorge Moran, O'Fallon, MO (US); Slobodan Trifunovic, Ankeny, IA (US); Lei Zhu, Ballwin, MO (US)

(21) Appl. No.: 17/590,095

(22) Filed: Feb. 1, 2022

Related U.S. Application Data
(60) Provisional application No. 63/146,408, filed on Feb. 5, 2021.

(51) Int. Cl. G16B 20/40; G16B 50/10; G16B 40/00; G16B 35/20

(52) U.S. Cl. CPC G16B 20/40; G16B 35/20; G16B 40/00; G16B 50/10

(57) ABSTRACT

The present disclosure provides methods for trait introgression involving crossing a donor plant comprising a trait of interest to an intermediate recurrent parent plant followed by crossing of the intermediate recurrent parent plant to one or more recipient parent plants. The present method may be used to introgression a desired trait of interest into the genome of an elite cultivar in fewer backcross generations, thereby accelerating new line production and reducing costs.

### REFERENCE TO RELATED APPLICATION

This application claims the benefit of U.S. Provisional Application No. 63/146,408, filed Feb. 5, 2021, which is herein incorporated by reference in its entirety.

### FIELD OF THE INVENTION

The present disclosure relates to the field of agricultural biotechnology, and more specifically to crop breeding and methods for improving trait introgression efficiency and operational efficiency.

### BACKGROUND OF THE INVENTION

Breeders are continually developing new cultivars through various plant genetic improvement programs. One critical step of these program is to modify highly specific genetic region or to add highly specific new traits. Such programs traditionally rely on introgression of alleles or genetic constructs conferring new or favorable traits from the genome of one line into the genome of an elite line though classic trait introgression methods. Such a trait integration method is pivotal in the success of genetic modified organism (GMO) of the late 1990s and the early 21 century. One must integrate the traits of interest precisely while in the meantime avoiding any negative impact on the overall genome and its phenotypic performance. Achieving such a dual goal successfully is non-trivial. Classic trait introgression methods, where each single elite line is converted individually and independently, are inefficient and costly. Due to the high cost, breeding programs normally wait until a sufficiently late stage in the new cultivar development in order to focus on a relative small number of elite lines for trait introgression process, normally after 2˜3 years of testing. This, together with the multiple generations and long time required for trait introgression, is a hindrance on rapid crop trait and trait-seed product development. As breeders look to accelerate crop variety development, it is critical to develop improved methods of trait introgression that increase efficiency and facilitate a faster generation of new cultivars.

### SUMMARY OF THE INVENTION

In one aspect, the present disclosure provides a method of deploying at least one trait of interest into a population of recipient parents, the method comprising a) grouping, by genetic distance, the population of recipient parents into at least one recipient parent group, wherein each recipient parent group comprises at least one intermediate recurrent parent centric in genetic distance relative to other members of the at least one recipient parent group; b) introgressing, through backcrossing, the at least one trait of interest from a donor parent to a first intermediate recurrent parent comprised in a first recipient parent group; and c) introgressing, through backcrossing, the at least one trait of interest from the first intermediate recurrent parent to other members of the first recipient parent group.

In one embodiment, the first recipient parent group is one of a plurality of recipient parent groups, wherein each member of the population of recipient parents is comprised within at least one of the plurality of recipient parent groups, and wherein the first intermediate recurrent parent is one of a plurality of intermediate recurrent parents, wherein each of the plurality of intermediate recurrent parents is comprised within at least one of the plurality of recipient parent groups. In another embodiment, the presently disclosed method further comprises introgressing the at least one trait of interest from the donor parent to each of the plurality of intermediate recurrent parents, and further introgressing the at least one trait of interest from each of the plurality of intermediate recurrent parents to other members of associated recipient parent groups.

In a further embodiment, the genetic distance between any two members of the at least one recipient parent group is at least 60% according to identity by decent. For instance, the genetic distance between any two members of the at least one recipient parent group may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity by decent. In other embodiments, the genetic distance between any two members of the at least one recipient parent group is at most 65% according to identity by descent. On yet other embodiments, the genetic distance between any two members of the at least one recipient parent group is at most 80% according to identity by descent.

In another embodiment, the at least one recipient parent group comprising the at least one intermediate recurrent parent further comprises up to ten other members of the population of recipient parents. In yet another embodiment, at least one member of the at least one recipient parent group is a member of a second, alternative and backup recipient parent group. In a further embodiment, the population of recipient parents consists of plants.

In one embodiment, the at least one trait of interest comprises at least one agronomic trait of interest. In another embodiment, the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

In another aspect, the present disclosure provides a system for deploying at least one trait of interest into a population of recipient parents for use in plant breeding, where the system comprises a) a breeding pipeline of a target environmental region with the genotype data of each of the inbred lines in the said pipeline; b) a computing device in communication with a data structure and configured to group, by genetic distance, the population of recipient parents into at least one recipient parent group, wherein each recipient parent group comprises at least one intermediate recurrent parent centric in genetic distance relative to other members of the at least one recipient parent group; c) a first introgression means, wherein the first introgression means is configured to introgress, through backcrossing, the at least one trait of interest from a donor parent to a first intermediate recurrent parent belonging to a first recipient parent group; and d) a second introgression means, wherein the second introgression means is configured to introgress, through backcrossing, the at least one trait of interest from the first intermediate recurrent parent to other members of the first recipient parent group; wherein a plant derived from at least one traited member of the population of recipient parents is planted in a growing space and directed into the breeding pipeline.

In one embodiment, the first recipient parent group is one of a plurality of recipient parent groups, wherein each member of the population of recipient parents is associated with at least one of the plurality of recipient parent groups, and wherein the first intermediate recurrent parent is one of a plurality of intermediate recurrent parents, wherein each of the plurality of intermediate recurrent parents is associated with at least one of the plurality of recipient parent groups. In another embodiment, the presently disclosed system further provides means for introgressing the at least one trait of interest from the donor parent to each of the plurality of intermediate recurrent parents, and further introgressing the at least one trait of interest from each of the plurality of intermediate recurrent parents to other members of associated recipient parent groups.

In a further embodiment, the genetic distance between any two members of the at least one recipient parent group is at least 60% according to identity by decent. For instance, the genetic distance between any two members of the at least one recipient parent group may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity by decent. In other embodiments, the genetic distance between any two members of the at least one recipient parent group is at most 65% according to identity by descent. On yet other embodiments, the genetic distance between any two members of the at least one recipient parent group is at most 80% according to identity by descent.

In another embodiment, the at least one recipient parent group comprising the at least one intermediate recurrent parent further comprises up to ten other members of the population of recipient parents. In yet another embodiment, at least one member of the at least one recipient parent group is a member of a second, alternative or backup recipient parent group. In still yet another embodiment, the at least one trait of interest is introgressed from the donor parent to the first intermediate recurrent parent through 2 or fewer backcrosses.

In one embodiment, the at least one trait of interest comprises at least one agronomic trait of interest. In another embodiment, the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

### BRIEF DESCRIPTION OF THE DRAWINGS

FIG. 1—Diagrammatical representation of different introgression methods. The panel on the left shows the conventional, point-to-point, classic trait introgression method and the two panels on the right show the introgression steps of the presently disclosed intermediate recurrent parent introgression method. Trait introgression via the intermediate recurrent parent method involves the steps of introgression of a trait of interest from a donor parent to an intermediate recurrent parent (IRP) in the first stage and introgression of the trait of interest from the intermediate recurrent parent (IRP) to recipient parent plants (RP) grouped with the intermediate recurrent parent.

FIG. 2—Timeline comparison between classic trait introgression method on the top line and intermediate recurrent parent (IRP) trait introgression methods on the bottom line. The classic trait introgression method contains 9 cycles, 3 cycles per year, to finish a trait conversion. Intermediate recurrent parent trait introgression method on the bottom line shows that this method allows trait introgression to begin as soon as the genotype data is available such that genetic distance can be estimated. In this example, being able to start conversion just one cycle earlier than normal and can save one year when developing new cultivars. The figure shows nursery planting cycles including new start (NS) planting, involving the planting of recipient parents and donor parents; F1progeny (F1) planting, involving the planting of the F1 progeny of the cross between the recipient parent and donor parent plants; first backcross (BC1 and BC1 IRP) planting, involving the planting of the progeny of the cross between the F1 plants and recipient parent or intermediate recurrent parent plants; second backcross (BC2 and BC2 IRP) planting, involving the planting of the progeny of the cross between the BC1 or BC1 IRP plants and the recipient parent or intermediate recurrent parent plants; third backcross (BC3 RP) planting, involving the planting of the progeny of the cross between the BC2 IRP plants and the recipient parent; zygosity (ZY) planting, involving the planning of the progeny of the selfing of the final backcross; increase (INC) planting, involving the planting of the seed resulting from the ZY planting, used to increase seed amount; hybrid make-up (HMU) planting, involving the planting of the seed resulting from the INC planting, used to create hybrid by crossing to lines of the opposite gender, for necessary hybrid crops, such as corn. The figure also includes flexible breeding cycles (indicated by “??”) during which additional breeding crosses may be conducted as necessary, such as conducting any additional selfing generations, stacking multiple traits together, or in the event of operational or environmental issues causing nursery failures, replanting of the previous nursery cycle as a reset.

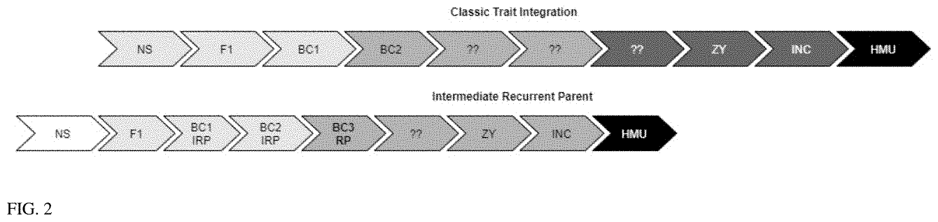

### DETAILED DESCRIPTION

The combination of desirable traits with an elite genome to generate new cultivars traditionally occurs through classic trait introgression methods. Classic trait introgression relies on a point-to-point trait distribution system, where a favorable or new trait from a donor plant is introgressed into a recipient plant through repeated backcrosses of the donor parent carrying the desired trait to a recurrent parent plant, for instance containing an elite or commercial genome. The final goal of the repeated backcrossing is to achieve a plant containing both the desired trait from the donor parent plant and a high recovery of the elite recurrent parent plant's genome to ensure the replication of performance of the elite recurrent parent plant. This final goal often requires an ultra-high identity by decent (IBD) and can therefore require multiple years to complete, even with the assistance of multiple cycles per year and winter nurseries or protected culture operations.

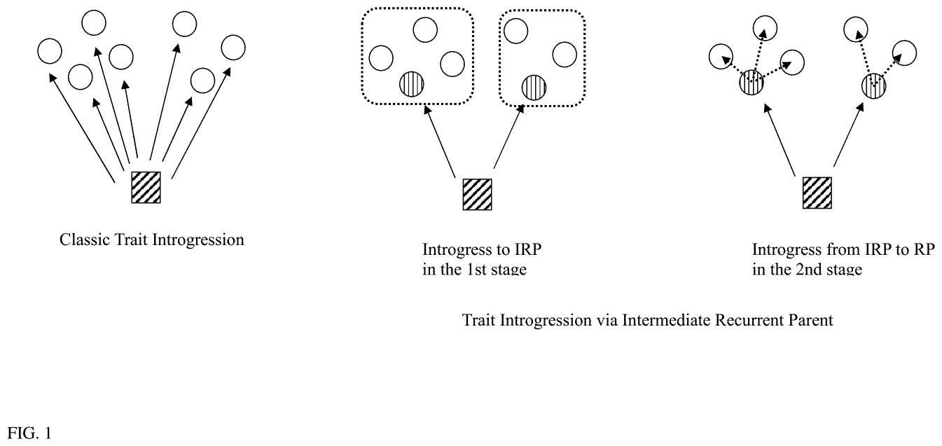

The present disclosure provides a novel method of developing new cultivars through a new method of trait introgression designed around a source-depot-edge pattern instead of the classic point-to-point system (FIG. 1). The presently disclosed method involves the use of an intermediate recurrent parent to significantly reduce the time required to develop new cultivars, reduce operational cost, and achieve the same level of probability of success as classic trait introgression methods. The presently disclosed trait introgression method therefore addresses the limitations of conventional methods through the use of an intermediate recurrent parent plant as an intermediate depot for the desired trait to be introgressed, which serves as a bridge between the source (or donor parent) and the edge (or the recipient parents).

### A. Advantages of the Intermediate Recurrent Parent Trait Introgression Method

In large scale commercial breeding operations, classic methods of trait introgression by marker-assisted backcrossing are extremely inefficient due to redundancy, repetition, time requirements, and high costs. For instance, classic trait introgression does not take into consideration the uncertainty of line fate within a breeding program. As a result, in a classic point-to-point distribution system, a large number of initial crosses may eventually be unnecessary if the recurrent parent involved is dropped during subsequent testing stage and line advancement. The necessity of starting classic trait introgression multiple years in advance of commercial launch results in having multiple line advancement stages during the whole trait introgression process, thus multiplying such line fate uncertainty.

Additionally, the genetic relationship of the recipient pool is not considered in the classic trait introgression method. Improving efficient and reducing redundancy by exploiting the existence of groups of highly similar lines was not previously considered. Pairwise genetic distance among lines of the same testing stage in most commercial breeding pipelines is seldom uniformly distributed. In a strict point-to-point distribution system, even for two or more genetically similar lines, individual sets of marker-assisted backcross projects are typically conducted for each line. By first converting a trait into an intermediate recipient, representing the shared genome of genetically similar lines, and then converting each individual line, the inefficiency of repetitive conversion of highly similar lines will be greatly reduced.

These two inefficiencies jointly limit the earliest opportunity to start classic trait introgression methods in a breeding program. While it is desirable to start classic trait introgression as early as possible to accelerate product launching, to improve the breeding cycling rate and genetic gain within a breeding program, but the sheer large number of lines can render such an option operationally and economically unfeasible.

Further, in an era of precision breeding and gene editing, the number of traits requiring introgression into new elite lines will continue to increase with the advances in understanding gene functions and discovery of new traits. The inefficiencies of a classic trait introgression system outlined above will limit the feasibility of converting a portfolio of well-defined, precisely engineered genomic regions to each new target line.

The presently disclosed method of intermediate recurrent parent trait introgression overcomes a number of inefficiencies of the classic trait introgression method.

### B. Grouping of Recipient Parents Plants and Selecting Intermediate Recurrent Parent Plants

The presently disclosed method facilitates the introgression of at least one desired trait through a source-depot-edge design, wherein the desired trait is introgressed from a donor plant initially to at least one intermediate recurrent parent plant and then from the intermediate recurrent parent plant to at least one recipient plant. The disclosed method involves several important design aspects including grouping of the plants within a breeding pipeline or population and selection of the intermediate recurrent parent plant within each grouping of plants.

The presently disclosed method involves the grouping of plants within a breeding pipeline or population of plants into which a desired trait is to be introgressed. The grouping may be done by any means known in the art to group plants, for instance, any means known to group plants by genetic distance, genetic relationship, or in more rudimentary form: general resemblance and breeder's perception. In certain embodiments of the disclosure, plants within a breeding pipeline or population can be grouped by genetic distance, for instance using identity by decent. In other, non-limiting embodiments, plants within a breeding pipeline or population can be grouped by pedigree, parentage, or any other mean known in the art that relates to genetic relationship or genetic distance.

In some embodiments of the disclosed method, the genetic distance between any two plants may be measured by percent sequence identity or percent identity by decent. For instance, genetic distance can be measured using any known sequence analysis techniques, including, but not limited to the use of genotype by sequencing, DNA fingerprinting, PCR-based detection methods (for example, TaqMan assays), microarray methods, mass spectrometry-based methods, and/or nucleic acid sequencing methods. Further, the methods or techniques discussed below regarding selection and detection of traits for introgression may also be used in some embodiments to estimate or determine the genetic distance between two or more plants.

In certain embodiments, plants can be grouped based on a predetermined or selected genetic distance threshold. This threshold can be selected or optimized, for instance, based on the breeding pipeline or population to be grouped. In some aspects, the grouping may be optimized such that the resulting grouping meets further design parameters, including for instance, the number of members in each group, and the total number of intermediate recurrent parent plants selected for the breeding pipeline or population, and the number of recipient parents to be included in more than one grouping. The plants within a breeding pipeline or population may be grouped into any number of necessary groups, including a single group or more than one group. For instance, a breeding pipeline or population may be grouped into 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more distinct groups of plants.

In one embodiment, grouping of plants may be done in-silico using a computing device. The in-silico grouping may be performed using any of the publicly available mathematic optimization computer programs suitable for creating such groupings, based for instance, on estimated genetic distance and pre-determined threshold. Multiple publicly or commercially available software platforms that are suitable are available to perform the in-silico calculations to determine recipient plant groupings and intermediate recurrent parent plants within each group, including but not limited to CPLEX, CBC, Gurobi, SCIP, and Xpress.

The presently disclosed method therefore provides, in some embodiments, a computing device in communication with a data structure and configured to group a population of plants, including recipient plants into which a desired trait of interest is to introgressed. For instance, the computing device may be configured to group a population of recipient plants in to distinct groups of plants based on genetic relationship thresholds. The computing device may further be configured to select at least one intermediate recurrent parent from each group of recipient parent plants, wherein the intermediate recurrent parent in each group is centric in genetic distance relative to other members of the group.

In some embodiments, at least one intermediate recurrent parent plant is selected from each of the groupings of recipient parent plants such that the remaining recipient parent plants are connected to at least one intermediate recurrent parent plant with a genetic distance no greater than a pre-determined threshold. In certain embodiments, the genetic distance between the members of the recipient parent groupings, for instance between each of the recipient parent plants in a grouping, or between the selected intermediate recurrent parent plant and the remaining recipient parent plants in the grouping, may be operationally discrete in nature, e.g., the genetic distance of a discrete number (one, two, three, four, five, six, etc.) of marker-assisted backcrosses. In one embodiment, a one-marker-assisted backcross equivalent distance means that if the recipient parent plant and the intermediate recurrent parent plant are within that distance threshold, backcrossing the intermediate recurrent parent plant to the recipient parent plant for a single backcross generation would be expected to bring the resulting progeny to a nearly indistinguishable level of performance to that of the recipient parent plant. The equivalent distance of one, two, three, or more backcrosses may for instance, be determined from historical classic trait introgression performance analysis and forward genetic simulation. In other embodiments, the genetic distance between the recipient parent plants in a group, or between the selected intermediate recurrent parent plant and recipient parent plants may be defined by identity by decent. For instance, in certain embodiments, the genetic distance may be defined as 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity by decent.

In one embodiment of the disclosure, a subset of one or more representative lines are selected from the population of elite lines into which at least one trait of interest is to be introgressed to serve as an intermediate recurrent parent plant. Once an intermediate recurrent parent plant is selected, an initial cross to the donor parent plant is performed resulting in at least one progeny plant comprising the desired trait from the donor parent plant. Backcrosses can then be made between a progeny plant comprising the desired trait from the donor parent plant and the selected intermediate recurrent parent plant. In certain embodiments, one or more backcrosses between the progeny plant and the selected intermediate recurrent parent plant can be performed to recover at least a portion of the intermediate recurrent parent genetic background or phenotypic performance. In some embodiments, the portion of the intermediate recurrent parent genetic background to be recovered is a portion sufficiently high enough to maintain a desired phenotypic performance, for instance to maintain a sufficiently high replication of phonotypic performance. For instance, one, two, three, four, or five backcrosses may be performed. In some embodiments, a complete recovery of the intermediate recurrent parent's genetic background or phenotypic performance is not required before crossing to the recipient parent plant may begin.

In other embodiments, introgression of at least one genetic locus or trait into an intermediate recurrent parent plant can be achieved through molecular genetic methods. Such molecular genetic methods include, but are not limited to, various plant transformation techniques and/or methods that provide for homologous recombination, non-homologous recombination, site-specific recombination, gene editing technology, and/or other genomic modification methods that provide for locus substitution or locus conversion.

After the at least one genetic locus or trait is introgressed into the intermediate recurrent parent plant, for instance after the desired number of backcrosses to the intermediate recurrent parent plant, the progeny comprising the trait can be crossed to a recipient parent plant. The recipient parent plant for this crossing is from the same population of elite lines from which the intermediate recurrent parent was selected, but is a different plant than the selected intermediate recurrent parent plant. In some embodiments, the progeny from this cross may be backcrossed to the recipient parent plant a sufficient number of crosses to achieve a resulting progeny plant that comprises the introgressed trait and high level of genetic background or performance recovery from the recipient parent plant. For instance, one, two, three, four, or five backcrosses may be performed. In certain embodiments, the desired high level of genetic background or performance recovery will be indistinguishable or nearly indistinguishable to that of the original recipient parent plant. In further embodiments, the final progeny from such backcrosses may be selfed or subjected to haploid doubling to develop an inbred plant.

In certain embodiments, when selecting intermediate recurrent parent plants, the carrying capacity of each intermediate recurrent parent plant, or number of recipient plants to which a single intermediate recurrent parent plant is crossed or connected, should be carefully designed, capped, and the sizes of different recipient parent groups should be balanced. For instance, during selection of the intermediate recurrent parent plants, the number of recipient parent plants each intermediate recurrent parent plant is allowed to connect to or cross with should be considered, and in certain embodiments, be constrained. Additionally, the number of connections per intermediate recurrent parent plant should be as uniform as possible. For instance, in breeding populations, it is not uncommon to see an unbalanced diversity distribution, where a large number of lines or varieties are closely related and concentrated around a number of historical key lines, while the remaining lines or varieties are not closely related. Allowing an intermediate recurrent parent plant to connect to or cross with an unconstrained number of recipient parent plants, as long as the distance threshold is satisfied will likely result in operational infeasibility, as in many crops the amount of pollen per intermediate recurrent parent plant may become a limiting factor. Therefore, in some embodiments of the disclosure, an intermediate recurrent parent plant can connect between 1 and 10 recipient parent plants. In other embodiments, an intermediate recurrent parent plant can connect to 1, 2, 3, 4, 5, 6, 7, 8, 9, 10, 11, 12, 13, 14, 15, 16, 17, 18, 19, 20, 25, 30, 35, 40, 45, or 50 recipient parent plants. In certain embodiments, the carrying capacity of each intermediate recurrent parent plant may thus be controlled and designed with the goal of staying below operation feasibility after taking into account the breeding population diversity and line drop rates.

In other embodiments, when selecting the intermediate recurrent parent plant, the redundancy of each intermediate recurrent parent plant may be maximized. Specifically, constraining the number of connections per intermediate recurrent parent plant to recipient parent plants, leads to an increase in the number of intermediate recurrent parent plants selected, particularly where there is a high level of diversity in a breeding population. In view of the increased number of intermediate recurrent parent plants, it is possible to increase the portion of recipient parent plants that can be connected to more than one intermediate recurrent parent plant, thus increasing the intermediate recurrent parent plant redundancy. As a result, the cases where a recipient parent plant is covered by or connected to only a single intermediate recurrent parent plant may be reduced. In some embodiments, most of the recipient parent plants can be connected to multiple intermediate recurrent parent plants that satisfy the required genetic distance constraints, thus increasing the redundancy such that the negative impact of a failure of a single intermediate recurrent parent plant introgression can be mitigated. In certain embodiments, this redundancy improves the operational reliability, creating flexibility of adjustment should any of the intermediate recurrent parent plants conversion fail.

As with the grouping of the breeding pipeline or population described above, in one embodiment of the disclosed method, the selection of intermediate recurrent parents may be done in-silico using a computing device. The presently disclosed method therefore provides, in some embodiments, a computing device in communication with a data structure and configured to select one or more intermediate recurrent parents based on the above described parameters and constraints, such that the intermediate recurrent parent in each group is centric in genetic distance relative to other members of the group. For instance, the computing device may be configured to select one or more intermediate recurrent parents based on genetic distance between the intermediate recurrent parent and the recipient parent plants in the same group, the carrying capacity of the intermediate recurrent parent or size of the group within which the intermediate recurrent parent is contained, and the redundancy of connections between recipient plants and the selected intermediate recurrent parent plants.

The in-silico selection of the intermediate recurrent parent may be performed using any of the publicly and commercially available mathematic optimization computer programs suitable for such selection, for instance, including but not limited to CPLEX, CBC, Gurobi, SCIP, and Xpress.

### C. Sources of Genetic Loci or Traits for Introgression

Genetic loci conferring traits for introgression from a donor parent may come from any source known in the art. For instance, in certain non-limiting embodiments, such genetic loci may be simply native genes, inherited genes, quantitative trait loci (QTL) that control quantitative expression of complex traits; or transgenes inserted into a recipient host plant or donor plant by a method of genetic engineering technologies, such as transformation or site-specific modification. Alternatively, the genetic modification may be by alternative engineering techniques, such as mutation, cloning, tilling, or other methods known to the art.

Desirable qualitative or agronomic traits include resistance to plant pathogens or pests, for example resistance to one or more of a viral disease, a bacterial disease, a fungal disease, a nematode disease and an insect pest. They may also be traits for tolerance to an herbicide, for example, inhibitors of 5-enolpyruvylshikimate-3-phosphate synthase (EPSPS), such as glyphosate; synthetic auxins, such as dicamba and 2,4-D; glutamine synthetase inhibitors, such as glufosinate; and acetyl CoA carboxylase (ACCase) inhibitors, such as quizalofop and haloxyfop. Other, non-limiting, desirable traits may include traits altering oil content or composition; water use efficiency; yield; drought resistance; seed quality; nutritional quality; nitrogen use efficiency; or tolerance to nitrogen stress.

In certain embodiments, donor parent plants may be selected on the basis of desirable qualitative or agronomic traits. The donor parent plant may contain one or more desirable trait for introgression. In some embodiments, the donor parent plant, intermediate recurrent parent plant, and recipient parent plant may be of the same taxa, while in others the donor parent plant, intermediate recurrent parent plant, and recipient parent plant may be of different but related taxa. Similarly, the donor parent plant, intermediate recurrent parent plant, or recipient parent plant each be an elite plant or cultivar, or the donor parent plant, intermediate recurrent parent plant, or recipient parent plant may a non-elite plant. In certain embodiments, optimization of donor parent plant choice can be done using techniques known in the art, for instance similar to those in classic trait introgression.

### D. Selection and Detection of Traits for Introgression

In some embodiments, the presently disclosed method for introgression of at least one desired trait includes introgression of a single trait of interest, or of more than one trait of interest. For instance, more than one trait of interest may be engineered to be introgressed into a narrow genetic region within a genome. The present disclosure therefore provides introgression of multiple traits of interest as a single heritable unit that will segregate together.

Where the at least one desired trait or trait of interest is a plant phenotype trait, selection for a desired trait may be by any of the ways known in the art, for example detecting or quantifying an expressed trait (selection criterion). In some cases, the trait of interest may be easily monitored by the presence or absence of a marker sequence known to be linked to the gene(s) controlling the trait of interest. This will be true in those cases where the trait has been introduced by a genetic modification to the donor parent. In other instances, the trait may be detected based on the phenotype. Any similar or other process for detecting the trait may therefore be used, as is known in the art.

In particular embodiments of the presently disclosed method, marker-assisted selection may be used to select backcross progeny, identify the trait of interest, or increase the efficiency any other step in the present method. Genetic markers that can be used in the practice of the presently disclosed method include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), DNA Amplification Fingerprinting (DAF), Sequence Characterized Amplified Regions (SCARs), Arbitrary Primed Polymerase Chain Reaction (AP-PCR), isozymes, and other markers known to those skilled in the art.

In certain embodiments of the disclosure, polymorphic markers can be used to detect a desired trait. Polymorphic markers may also serve as useful tools for assaying plants for determining the genetic distance or degree of identity between lines or varieties. For instance, polymorphic markers can assist in determining the degree of identity by decent between lines or varieties used as donor plants, intermediate recurrent parent plants, or recipient plants.

Nucleic acid-based analyses for determining the presence or absence of the genetic polymorphism (i.e. for genotyping) can be used in the method of the present disclosure. A wide variety of genetic markers for the analysis of genetic polymorphisms are available and known to those of skill in the art. The analysis may be used to identify or select for desired traits, or in certain embodiments to identify the genetic distance, for instance the degree of identity by decent, between plants in a population.

As used herein, nucleic acid analysis methods include, but are not limited to, genotype by sequencing, DNA fingerprinting, PCR-based detection methods (for example, TaqMan assays), microarray methods, mass spectrometry-based methods and/or nucleic acid sequencing methods. In certain embodiments, the genetic distance between plants within a population, such as the genetic distance between a donor plant and an intermediate recurrent parent plant, or between an intermediate recurrent parent plant and a recipient plant, may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. (1986) Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entirety. However, the compositions and methods of the presently disclosed method can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to locus-specific oligonucleotide (ASO) probes as disclosed in U.S. Pat. Nos. 5,468,613 and 5,217,863. 5,468,613 discloses locus specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus interest, for instance conferring a trait interest, or the genome of plants useful in the presently disclosed method, can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

### Definitions

For purpose of clarity in reading the following specification and appended claims, the following terms and expressions shall have the meanings provided, wherein:

As used herein a “trait,” “desired trait,” “trait of interest,” or “trait of agronomic interest,” refers to a phenotype conferred by a particular allele, gene, or grouping of genes at a locus or loci in the genome of a plant. In certain embodiments, a trait of the present disclosure may be a trait related to suitability for a crop end-use or may be a trait that provides a commercial value. A trait of the present disclosure may comprise, but is not limited to, herbicide tolerance, insect control, increased plant pathogen resistance, enhanced oil composition, enhanced oil content, increased water use efficiency, increased yield, increased drought resistance, increased seed quality, improved nutritional quality, increased nitrogen use efficiency, or tolerance to nitrogen stress.

As used herein, a “locus,” or “genetic locus” refers to fixed position on a genomic sequence. The term “loci” is the plural form of the term “locus.” A locus may refer to a nucleotide position at a reference point on a chromosome, such as a position from the end of the chromosome. A locus may comprise genetic material, including but not limited to a genetic marker, or a gene, such as a transgene, or a native gene.

As used herein, an “allele” refers to one or more alternative forms of a genomic sequence at a given locus on a chromosome. In a diploid cell or organism, the two alleles of a given gene occupy corresponding loci on a pair of homologous chromosomes. Two or more alleles constitutes a polymorphism. The polymorphic sites of any nucleic acid sequence can be determined by comparing the nucleic acid sequences at one or more loci.

As used herein, a “marker” refers to a detectable characteristic that can be used to discriminate between alleles or organisms. Examples of such characteristics include, but are not limited to, genetic markers.

As used herein, the term “genotype” refers to the specific allelic makeup of a plant.

As used herein, the term “phenotype” refers to the detectable characteristics of a cell or organism, which characteristics are the manifestation of gene expression and thus influenced by genotype.

As used herein, “identity by decent” refers to the sequence identity or similarity between two or more individual that is result of genetic inheritance, or inheritance of the similar nucleotide sequence from a common ancestor, or the portion of genomic segments that is shared between two individuals. In certain embodiments, plants or genomes of the present disclosure may share an identity by decent as defined by a percentage of sequence identity that is derived from a common ancestor.

As used herein, the term “genetic distance” refers to the sequence similarity between the genome of two or more plants. The genetic distance between two or more plants may be defined, in certain embodiments, by the number of marker-assisted backcrosses required to recover, or essentially recover, the genome or the level of agronomic performance of one of the plants in the backcross. For example, a one marker-assisted backcross equivalent distance means that if two plants are within that distance threshold, backcrossing one of the plants to the other for a single backcross generation would be expected to bring the resulting progeny to a nearly indistinguishable level of performance to that of the backcrossed parent plant. Genetic distance may also be measured, in certain embodiments, by percent sequence identity or percent identity by decent.

As used herein, the term “plant” includes plant cells, plant protoplasts, plant cells of tissue culture from which a plant can be regenerated, plant calli, plant clumps and plant cells that are intact in plants or parts of plants. Non-limiting examples of plant parts include embryos, pollen, ovules, seeds, leaves, stems, flowers, branches, fruit, kernels, ears, cobs, husks, stalks, roots, root tips, anthers, and the like. Plants of the current disclosure include any plant species, including monocots or dicots, and may, in certain embodiments, include any crop plant, for instance forage crops, oilseed crops, grain crops, vegetable crops, fiber crops, and turf crops. In other embodiments, plant of the current disclosure may include, but are not limited to, corn (maize) (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), wheat (Triticum aestivum), soybean (Glycine max), tobacco (Nicotiana tabacum), cotton (Gossypium barbadense, Gossypium hirsutum), oats, barley, and vegetables.

As used herein, the term “population” refers to a grouping of one or more plants. In certain embodiments, a population of plants comprises at least about 10, 50 100, 250, 500, 1,000, 5,000, 10,000, 50,000, or 100,000 or more plants.

As used herein, the term “breeding pipeline” refers to a population of germplasm or plants to be used in a breeding program to generate new cultivars.

As used herein, the terms “variety” or “cultivar” refers to a group of similar plants that by their genetic pedigrees and performance can be identified from and are distinct from other varieties or cultivars within the same species.

As used herein, “elite variety” or “elite cultivar” refers to a variety that has resulted from breeding and selection for superior agronomic performance. As used herein, the term “elite line” refers to a line that results from breeding and selection for superior agronomic performance. An “elite plant” refers to a plant belonging to an elite variety or elite line. Similarly, an “elite germplasm” or elite strain of germplasm is an agronomically superior germplasm. As used herein an “elite genome” refers to the genome of an elite plant.

As used herein, the term “introgressed” or “introgression,” when used in reference to a genetic locus, or trait conferred by a genetic locus, refers to a genetic locus or trait that has been introduced into a new genetic background, such as through backcrossing. As used herein, “trait introgression” refers to the introgression of a genetic locus that confers a trait. Introgression of a genetic locus or trait can be achieved through plant breeding methods, such as those of the present disclosure, and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the term “classic trait introgression” refers to the traditional method of introgression of a trait of interest or a locus conferring a trait of interest from the genome of a donor plant into the genome of each recipient plant. Classic trait introgression traditionally relies on repeated backcrosses of the donor parent carrying the desired trait to recurrent parent plants, for instance containing an elite or commercial genome. The final goal of the repeated backcrossing is to achieve a plant containing both the desired trait from the donor parent plant and a high recovery of the recurrent parent plant's genome to ensure performance recovery of the elite recurrent parent plant.

As used herein, the term “donor parent” refers to a plant that contains a trait of interest or locus conferring a trait of interest in its genome for introgression into a recipient plant. The donor parent may be a homozygous (inbred), or a heterozygous (hybrid) plant, and may be of the same or a related taxa to the recipient parent.

As used herein, the term “recipient parent” refers to a plant into which a trait of interest or a locus conferring a trait of interest will be introgressed. In certain embodiments, a recipient plant may be an elite cultivar or comprise an elite genome. In some embodiments, a recipient parent may be used as a recurrent parent in the presently disclosed invention.

As used herein, the term “recurrent parent” refers to a plant into which a trait of interest or a locus conferring a trait of interest will be introgressed and which is used for at least one backcross during a trait introgression method. In certain embodiments, a recurrent plant may be an elite cultivar or comprise an elite genome. In some embodiments, a recurrent parent is a homozygous (inbred) plant.

As used herein, the term “intermediate recurrent parent” refers to a plant selected from a group or population of recipient parent plants for crossing to a donor parent plant, where the remaining recipient parent plants are connected to at least one intermediate recurrent parent plant with a genetic distance no greater than a pre-selected threshold. In certain embodiments, the genetic distance between the selected intermediate recurrent parent plant and a recipient parent plant may be operationally discrete in nature, e.g., the genetic distance of a discrete number (1, 2 or 3, etc.) of marker-assisted backcrosses. In other embodiments, the genetic distance between the selected intermediate recurrent parent plant and recipient parent plants may be defined by identity by decent. For instance, in certain embodiments, the genetic distance may be defined as 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity by decent.

As used herein, the term “backcrossing” refers to a process in which a breeder repeatedly crosses progeny, for instance hybrid progeny, such as a first generation hybrid (F₁), back to one of the parents of the hybrid progeny. Backcrossing can be used to introduce one or more loci, traits, or transgenes of interest from one genetic background into another and/or to recover the genome or agronomic performance or phenotype of one of the parents of the hybrid progeny.

As used herein, the term “crossing” refers to the mating of two parent plants.

As used herein, the term “marker-assisted breeding” or “marker-assisted selection” refers to a breeding or selection process where a trait or phenotype of interest is selected based on a marker, such as a genetic marker, linked to a trait or phenotype of interest, rather than selection of the trait or phenotype itself.

As used herein, the term “marker-assisted backcross” refers to a method of breeding where a trait or phenotype of interest is selected based on a marker, such as a genetic marker, linked to a trait or phenotype of interest, where the selected plant is backcrossed to one of its parent plants.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### EXAMPLES

The following examples are included to more fully describe the invention. It should be appreciated by those of skill in the art that many modifications can be made to the specific examples which are disclosed to obtain similar results. Any such modifications apparent to those skilled in the art are deemed to be within the scope of the invention.

### Example 1

### Grouping Recipient Parents and Selecting Intermediate Recurrent Parents

Plants within a population into which a desired trait is to be introduced are grouped into distinct recipient parent groups using the following steps. Initially, a genetic distance matrix (D) is generated for all of the recipient plants within the population (N). The resulting matrix will be the size of N×N, where each element in the matrix represents the genetic distance between each pair of recipient parents in a breeding pipeline. For example:

$$\begin{matrix}D = [ [ 62 , 52 , 58 , … , 81 , 26 , 62 ] , \\ [ 69 , 52 , 29 , … , 82 , 53 , 91 ] , \\ [ 61 , 94 , 91 , … , 25 , 66 , 50 ] , … , \\ [ 28 , 74 , 83 , … , 46 , 34 , 60 ] , \\ [ 70 , 49 , 72 , … , 97 , 68 , 77 ] , \\ [ 98 , 99 , 87 , … , 74 , 39 , 26 ] ]\end{matrix}$$

A genetic distance threshold is established for the specific population, defined for instance, by identity by decent or number of discrete backcross generations. A genetic simulation is run applying the determined genetic distance threshold, such that it is determined for every combination of two lines within the population whether the pair meets the threshold. This will determine for each pair whether they are genetically similar enough (meets or exceeds the genetic distance threshold) to be allowed to be grouped together. This information is used to create a new binary N×N matrix (D′), where the integer “1” is used in the matrix to represent pairs of plants that meet or exceed the genetic distance threshold and thus are allowed to be grouped together, and the integer “0” is used in the matrix to represent pairs that do not meet the genetic distance threshold and thus cannot be grouped together. For example:

$$\begin{matrix}{D}^{′} = [ [ 1 , 0 , 0 , … , 1 , 0 , 0 ] , \\ [ 0 , 0 , 0 , … , 1 , 0 , 1 ] , \\ [ 0 , 1 , 1 , … , 0 , 0 , 0 ] , … , \\ [ 0 , 0 , 1 , … , 0 , 0 , 0 ] , \\ [ 0 , 0 , 0 , … , 1 , 0 , 0 ] , \\ [ 1 , 1 , 1 , … , 0 , 0 , 0 ] ]\end{matrix}$$

Based on the binary matrix (D′), an array of decision variables for selection as an intermediate recurrent parent (I), of N length, is generated. The resulting variables are binary, such that “1” represents a line that is allowed to group with one or more than one other lines, and thus may be selected as an intermediate recurrent parent, and “0” represents a line that is allowed to connect one only one other line, and thus cannot be selected as an intermediate recurrent parent.

Another array of decision variables, this time for number of connections (C), is defined. This C array is N in length, with each member representing a single line. The variables in the connections array will be integer variables with an upper bound of C_{ub}, such that for each line in the population of N the corresponding element in the intermediate recurrent parent array and the connections array satisfies the following parameters:

$$\{ \begin{matrix}I = 0 C = 1 \\ I = 1 {C}_{ab} \geq C \geq 1\end{matrix}$$

A further array of decision variables, for redundancy (R), is generated. Using the second matrix created (the D′ matrix), the rows of the matrix are sliced, where the corresponding value of I=1, the column sum of the resulting slice of D′ equals the R array of decision variables. For example:

$$\begin{matrix}{D}^{′} slice = [ [ 1 , 0 , 0 , … , 1 , 0 , 0 ] , \\ [ 0 , 0 , 0 , … , 1 , 0 , 1 ] , \\ [ 0 , 1 , 1 , … , 0 , 1 , 0 ] ]\end{matrix}$$

$$R = [ 1 , 1 , 1 , … , 2 , 1 , 1 ]$$

Two optimization objectives are created based on the above arrays, as follows: 1) minimize the summation of I (the number of IRPs), and 2) maximize the count of the larger-than-one elements in R (the redundancy of IRPs).

A mixed-integer linear programming model is then created based on the pre-determined group size and genetic distance constraints and decision variables described above, and mathematic optimization software is used to solve for the solution, or in practical terms, to group the population of recipient parent plants into distinct groups meeting the pre-determined group size and genetic distance thresholds and select intermediate recurrent parent plants within each group. Multiple publicly or commercially available software platforms are available to perform the grouping and intermediate recurrent parent selection, including but not limited to CPLEX, CBC, Gurobi, SCIP, and Xpress.

### Example 2

### Developing New Cultivars Using Intermediate Recurrent Parents

Breeding experiments were conducted to test and validate the presently disclosed intermediate recurrent parent trait introgression method. The experiments were designed around conducting two backcrosses between intermediate recurrent parent plants and donor parent plants, followed by subsequent backcrosses to final recipient parent plants for no more than 2 generations.

Intermediate recurrent parent plants were selected using the methods described in Example 1, such that the remaining recipient parents shared no more than one backcross distance to the selected intermediate recurrent parent plants. Limiting the genetic distance between the selected intermediate recurrent parent plants and the recipient parent plants to no more than one backcross provides a great advantage over the genetic distance between the donor parent plant and the recipient parent plant achievable through classic trait introgression methods. For instance, in classic trait introgression methods, donors are typically either transformational germplasm, which is genetically very distant from the recipient parent plant, or recently finished conversions from a classic trait introgression, which have base germplasm several years older than the current breeding cohort. Due to the continuous breeding effort over the years, those donors will not be able to provide a genetic distance between the donor parent plant and recipient parent plant of no more than one backcross consistently.

Specifically, approximately 600 pre-commercial inbred corn plants were divided into two groups with comparable genetic diversity using the methods described in Example 1. Trait integration was performed on both groups, one group though classic trait integration and the other group through intermediate recurrent parent integration.

The group of plants for intermediate recurrent parent integration was configured such that the intermediate recurrent parent plant to recipient parent plant distance was controlled at 80% identity by decent or better and each intermediate recurrent parent group was capped at 10 members in size including the intermediate recurrent parent plant itself. Two backcrosses were performed between the intermediate recurrent parent plant and donor parent plant for each intermediate recurrent parent plant. After backcrossing intermediate recurrent parent to donor parent for 2 generations, one or two generations of backcrosses between the now intermediate backcross 2 generation plant and each recipient parent plant in the same intermediate recurrent parent group were performed as needed to achieve at least 90% recipient parent recovery identity by decent.

During the first year of trait introgression, due to on-going parallel field trial and selection, 25 lines from each group (classic trait integration group and intermediate recurrent parent trait integration group) were selected for the next stage of field trials due to having demonstrated superior performance in the field trial. The lines not selected were therefore dropped from trait conversion process accordingly.

When trait integration for the classic trait integration group and intermediate recurrent parent trait integration group was compared side-by-side, the final conversion quality was defined as percent recipient genome recovered, measured by identity by decent, and conversion success was defined as the ability to obtain equal or higher than the quality standard within 9 generations including selfing and seed increase nursery generations. A successful conversion was defined as one resulting in a line homozygous for the trait of interest. A successful conversion also must produce at least one single ear with more than 200 kernels from the increase nursery.

After selection for the next stage of field trials, the conversion quality and success of the remaining groups of 25 lines in each trait integration method group was compared. Due to having different commercial target and associated target strategies for different lines or gender, each single line often has multiple conversions for different traits, and the exact number varies among the inbreds These resulted in 66 conversions and 50 conversions for 25 inbreds in the classic trait integration group and intermediate recurrent parent group, respectively. Out of the 66 conversions for the classic trait integration group, 2 were technical failures due to low identity by decent, with 9 classified as at-risk due to having only borderline recipient parent identity by decent recovery and/or low kernel counts. Out of the 50 conversions for the intermediate recurrent parent group, there were no failures and only 3 classified as at-risk, having only borderline recipient parent identity by decent recovery and/or low kernel counts.

A 2×2 contingency table test of the result above, treating both at-risk and failed conversions as the undesired state and successful conversion as the desired state, yield a Pearson Chi-square without correction for continuity of 3.05 (P=0.0807). Therefore, it can be concluded that there is no significant difference between intermediate recurrent parent and classic trait integration conversion methods at an alpha level of (P<0.05). The intermediate recurrent parent integration method therefore can be said to be at least as reliable as classic trait integration method.

During the experiments, it was observed that when the intermediate recurrent parent plant to recipient parent plant genetic distance requirement was satisfied, on average one intermediate recurrent parent plant was required out of about six pre-commercial 1 inbreds, The reduction ratio of 1:6, greatly reducing the number of projects compared with classic trait introgression methods. While a classic approach would start 6 conversions for 6 inbreds, IRP approach only need to start 1. The experiments also confirmed that it is possible to successfully execute IRP with each intermediate recurrent parent plant designed to carry at most 9 to 12 recipient parent plants. After pre-commercial 2 advancement, most of the 9˜12 lines per IRP group were dropped from the pipeline and most intermediate recurrent parent plants were only required to carry one recipient parent plant, and as a result, an intermediate conversion plants only need to produce enough seed/pollen for 1 final conversion, instead of all 9˜12 lines it is designed to cover. Indirectly, this demonstrates the benefit of the grouping method's provision for high redundancy, such that recipient plant may be included in more than one group and thus connected to more than a single intermediate recurrent parent, and as a result the risk of heavily relying on a few key intermediate recurrent parent plants was largely avoided.

During the design phase, it was estimated from genetic simulation that 46% of the projects from the intermediate recurrent parent covering group would be successful with two backcrosses to the intermediate recurrent parent plant and one backcross to the recipient parent plant, while the remaining 54% projects would likely require an additional backcross to the recipient parent plant. This was validated with an observed ratio of 45.7% to 54.3%. From an operational standpoint both categories were successful as both will satisfy target product delivery operation timeline, thus the practical probability of success is 100%.

Additionally, as selection of the intermediate recurrent parent plants was performed using only genotypic data and not requiring the accumulation of multi-stage and multi-year phenotype data, intermediate recurrent parent method could thus be executed in a very early stage without any field-based performance testing and screening, these experiments demonstrate that it is entirely feasible to start the present intermediate recurrent parent trait introgression method much earlier in screen stages than classic trait introgression methods. Such an approach is expected to require converting approximately 200 intermediate recurrent parent plants per relative maturity grouping to represent screen stage lines in the order of 10⁵. It is expected that implementing the present intermediate recurrent parent trait introgression strategy can accelerate new line development. Each year of acceleration of new line development provides a huge economic value.

### Example 3

### Conversion of Female Inbreds

Both the regular TI conversions method, and the new IRP method were applied to convert female inbreds of a particular corn breeding pipeline. Both methods completed a conversion process within a similar timeframe, and the final conversions were genotyped and the percentage of RP recovery was estimated by a haplotype-based identity-by-descent method. The difference between the two methods is statistically insignificant (Welch's test of unequal variance, t=0.568, two-tailed p=0.574).

TABLE 1
Descriptive statistics and quantiles of
conversion quality as measured by IBD

| Method | Count | Mean | Std | Min | 25% | 50% | 75% | Max |
|---|---|---|---|---|---|---|---|---|
| Regular TI | 15 | 94.76 | 5.92 | 76.59 | 93.18 | 97.70 | 98.01 | 98.90 |
| IRP | 19 | 93.56 | 6.35 | 69.72 | 92.52 | 94.47 | 97.25 | 98.83 |

Additionally, data for eight traits associated with the two converted populations are presented below. The data below lack an indicator for confounding factors of different performance of the base-inbreds in the two groups which might make one process more successful than the other during the conversion process. Thus, IRP may at least be used produce results comparable to more traditional TI methods.

TABLE 2
Data for eight traits associated with two converted populations

| | | p-value | |
|---|---|---|---|
| | Welch's t | (2 tail) | |
| YLD_BE_G | 1.616 | 0.200 | Yield |
| MST_BE_G | 0.000 | 1.000 | Moisture |
| TWT_BE_G | 0.441 | 0.679 | Tassel Weight |
| STLP_G | 1.206 | 0.305 | Stalk-lodged Percent |
| RTLP_G | −0.752 | 0.508 | Root-lodged Percent |
| PHT_G | 1.538 | 0.187 | Plant Height |
| EHT_G | 0.851 | 0.473 | Ear Height |
| ERM_G | −1.284 | 0.263 | Estimated Relative Maturity |

The method described above therefore may enable a significant increase in breeding pipeline capacity without incurring costs, both monetary and in terms of needed space. For example, the capacity of a corn breeding pipeline could be doubled without significant expense and without the need for additional space. In addition, the method described above may reduce the cost of a breeding pipeline, both monetary and in terms of needed space.

What is claimed is:

1. A method of deploying at least one trait of interest into a population of recipient parents, the method comprising:
a) grouping, by genetic distance, the population of recipient parents into at least one recipient parent group, wherein each recipient parent group comprises at least one intermediate recurrent parent centric in genetic distance relative to other members of the at least one recipient parent group;
b) introgressing, through backcrossing, the at least one trait of interest from a donor parent to a first intermediate recurrent parent comprised in a first recipient parent group; and
c) introgressing, through backcrossing, the at least one trait of interest from the first intermediate recurrent parent to other members of the first recipient parent group.

2. The method of claim 1, wherein the first recipient parent group is one of a plurality of recipient parent groups, wherein each member of the population of recipient parents is comprised within at least one of the plurality of recipient parent groups, and wherein the first intermediate recurrent parent is one of a plurality of intermediate recurrent parents, wherein each of the plurality of intermediate recurrent parents is comprised within at least one of the plurality of recipient parent groups.

3. The method of claim 2, further comprising introgressing the at least one trait of interest from the donor parent to each of the plurality of intermediate recurrent parents, and further introgressing the at least one trait of interest from each of the plurality of intermediate recurrent parents to other members of associated recipient parent groups.

4. The method of claim 1, wherein a genetic distance between any two members of the at least one recipient parent group is at most 65% according to identity by descent.

5. The method of claim 1, wherein a genetic distance between any two members of the at least one recipient parent group is at most 80% according to identity by descent.

6. The method of claim 1, wherein the at least one recipient parent group comprising the at least one intermediate recurrent parent further comprises up to ten other members of the population of recipient parents.

7. The method of claim 1, wherein at least one member of the at least one recipient parent group is a member of a second recipient parent group.

8. The method of claim 1, wherein the population of recipient parents consists of plants.

9. The method of claim 1, wherein the at least one trait of interest comprises at least one agronomic trait of interest.

10. The method of claim 9, wherein the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

11. A system for deploying at least one trait of interest into a population of recipient parents for use in plant breeding, the system comprising:
a) a breeding pipeline of a target environmental region;
b) a computing device in communication with a data structure and configured to group, by genetic distance, the population of recipient parents into at least one recipient parent group, wherein each recipient parent group comprises at least one intermediate recurrent parent centric in genetic distance relative to other members of the at least one recipient parent group;
c) a first introgression means, wherein the first introgression means is configured to introgress, through backcrossing, the at least one trait of interest from a donor parent to a first intermediate recurrent parent belonging to a first recipient parent group; and
d) a second introgression means, wherein the second introgression means is configured to introgress, through backcrossing, the at least one trait of interest from the first intermediate recurrent parent to other members of the first recipient parent group;
wherein a plant derived from at least one traited member of the population of recipient parents is planted in a growing space and directed into the breeding pipeline.

12. The system of claim 11, wherein the first recipient parent group is one of a plurality of recipient parent groups, wherein each member of the population of recipient parents is associated with at least one of the plurality of recipient parent groups, and wherein the first intermediate recurrent parent is one of a plurality of intermediate recurrent parents, wherein each of the plurality of intermediate recurrent parents is associated with at least one of the plurality of recipient parent groups.

13. The system of claim 12, further comprising introgressing the at least one trait of interest from the donor parent to each of the plurality of intermediate recurrent parents, and further introgressing the at least one trait of interest from each of the plurality of intermediate recurrent parents to other members of associated recipient parent groups.

14. The system of claim 11, wherein a genetic distance between any two members of the at least one recipient parent group is at most 65% according to identity by descent.

15. The system of claim 11, wherein a genetic distance between any two members of the at least one recipient parent group is at most 80% according to identity by descent.

16. The system of claim 11, wherein the at least one recipient parent group comprising the at least one intermediate recurrent parent further comprises up to ten other members of the population of recipient parents.

17. The system of claim 11, wherein at least one member of the at least one recipient parent group is a member of a second recipient parent group.

18. The system of claim 11, wherein the at least one trait of interest is introgressed from the donor parent to the first intermediate recurrent parent through 2 or fewer backcrosses.

19. The system of claim 11, wherein the at least one trait of interest comprises at least one agronomic trait of interest.

20. The system of claim 19, wherein the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.
